(19) United States
(12) Patent Application Publication
(10) Pub. No.: US 2010/0150912 A1
(43) Pub. Date: Jun. 17, 2010

(54) BLOCKING INTERACTION BETWEEN PATHOGEN FACTORS AND FACTOR H TO INHIBIT HEMORRHAGIC SYNDROMES

(75) Inventor: Rino Rappuoli, Castelnuovo Berardenga (IT)

Correspondence Address: NOVARTIS VACCINES AND DIAGNOSTICS INC., INTELLECTUAL PROPERTY- X100B, P.O. BOX 8097, Emeryville, CA, 94662-8097, US

(73) Assignee: NOVARTIS AG, Basel (CH)

(21) Appl. No.: 12/450,697

(22) PCT Filed: Apr. 11, 2008
(86) PCT No.: PCT/IB2008/001765

§ 371 Date: Oct. 6, 2009

Related U.S. Application Data
(60) Provisional application No. 60/922,997, filed on Apr. 11, 2007.

(51) Int. Cl. A61K 39/395; A61K 39/40; A61K 39/42; C07K 16/08; C07K 16/12; A61K 38/16

(52) U.S. Cl. 424/133.1; 424/164.1; 424/159.1; 530/388.3; 530/388.4; 530/387.3; 514/12

(57) ABSTRACT

If pathogen factors such as meningococcal NMB 1870 or flaviviral NS1 are able to sequester factor H in the blood then its inhibitory effect on complement may be disturbed, thereby permitting C3 to initiate a dramatic attack on host endothelial tissue. In combination with a strong inflammatory response, this attack can result in sever damage to the endothelium, with resulting hemorrhagic syndrome. Blocking the interaction between pathogen factors and factor H may thus be used to treat and/or prevent these pathogen-induced hemorrhagic syndromes. The interaction may, for instance, be blocked by antibodies, either delivered endogenously (passive immunisation) or produced by a patient's immune system (active immunisation).

This invention relates to prevention and treatment of hemorrhagic diseases.

### BACKGROUND ART

Dengue virus and West Nile virus are both flaviviruses. One symptom of Dengue virus infection can be a fatal hemorrhagic syndrome (DHF: dengue hemorrhagic fever). West Nile virus has also been reported to cause fatal hemorrhagic fever [1]. A similar hemorrhagic syndrome can result from bacterial infection by Neisseria meningitidis (meningococcus).

The severity and mechanism of these hemorrhagic fevers is currently unexplained.

There is thus a need for ways of treating and/or preventing such hemorrhagic fevers.

### DISCLOSURE OF THE INVENTION

It has been observed that patients with complement deficiency (in C3 or C6) are particularly susceptible to meningococcal infections, but that these infections are never severe and do not cause hemorrhagic fever.

Meningococcal protein ‘NMB1870’ has been reported [56] to bind to the human complement protein Factor H (“fH”). West Nile virus non-structural protein 1 (NS1) also binds to fH [2]. The fH plasma protein binds to complement protein C3b. It is the dominant complement control protein, and in its absence the regulation of complement activation breaks down completely. When functioning normally, it prevents complement (driven by C3) from attacking host tissue.

If pathogen factors such as NMB1870 or NS1 are able to sequester fH in the blood then its inhibitory effect on complement may be disturbed, thereby permitting C3 to initiate a dramatic attack on host endothelial tissue. In combination with a strong inflammatory response, this attack can result in sever damage to the endothelium, with resulting hemorrhagic syndrome.

Blocking the interaction between pathogen factors and fH may thus be used to treat and/or prevent these pathogen-induced hemorrhagic syndromes. The interaction may, for instance, be blocked by antibodies, either delivered endogenously (passive immunisation) or produced by a patient's immune system (active immunisation). Other antagonists of the interaction may also be used.

Thus the invention provides a method for treating a hemorrhagic syndrome caused by a pathogen in a patient, comprising a step of administering to the patient a medicament that prevents the interaction between Factor H and a pathogen factor.

The invention also provides a non-murine monoclonal antibody that binds to a pathogen factor, wherein the pathogen factor can bind to human Factor H. The antibody will typically be a humanised or human antibody, and can inhibit the binding interaction between the pathogen factor and Factor H. The antibodies can be used to treat patients.

Where the pathogen is meningococcus, the pathogen factor is NMB1870. Where the pathogen is West Nile virus or Dengue virus, the pathogen factor is the viral NS1 protein.

The invention also provides a method for preventing or treating a hemorrhagic syndrome caused by a pathogen, comprising a step of administering to a patient a medicament comprising a protein sharing an epitope with a pathogen factor that can bind to Factor H. An immune response raised against the epitope can block the ability of a pathogen factor to sequester fH.

The invention also provides a method for preventing or treating meningococcal disease in a patient, comprising a step of administering to the patient a NMB1870 protein, wherein the patient has a complement system with a functional C3 component.

The invention also provides a method for preventing or treating meningococcal disease in a patient, comprising a step of administering to the patient a NMB1870 protein and at least one other meningococcal immunogen, wherein the patient has a complement system with a deficient C3 component.

The invention also provides a method for preventing or treating West Nile virus disease in a patient, comprising a step of administering to the patient a West Nile virus NS1 protein, wherein the patient has a complement system with a functional C3 component.

The invention also provides a method for preventing or treating West Nile virus disease in a patient, comprising a step of administering to the patient a West Nile virus NS1 protein and at least one other West Nile virus immunogen, wherein the patient has a complement system with a deficient C3 component.

The invention also provides a method for preventing or treating Dengue virus disease in a patient, comprising a step of administering to the patient a Dengue virus NS1 protein, wherein the patient has a complement system with a functional C3 component.

The invention also provides a method for preventing or treating Dengue virus disease in a patient, comprising a step of administering to the patient a Dengue virus NS1 protein and at least one other Dengue virus immunogen, wherein the patient has a complement system with a deficient C3 component.

Thus the invention also provides a method for preventing or treating a hemorrhagic syndrome caused by a pathogen in a patient, comprising a step of administering to the patient a Factor H protein or a Factor H protein decoy.

### Antibodies

The invention provides a method for treating a hemorrhagic syndrome by administering a medicament that prevents the interaction between Factor H and a pathogen factor. The active ingredient in the medicament may be an antibody. Suitable antibodies can recognise the pathogen factor and may inhibit its binding interaction with Factor H. Antibodies against meningococcal NMB1870, West Nile virus NS1 and dengue virus NS1 are already known in the art.

Antibodies of the invention may take various forms, but preferred antibodies are human antibodies. Unlike non-human antibodies, human antibodies will not elicit an immune response directed against their constant domains when administered to humans. Moreover, their variable domains are 100% human (in particular the framework regions of the variable domains are 100% human, in addition to the complementarity determining regions [CDRs]) and so will not elicit an immune response directed against the variable domain framework regions when administered to humans. The human antibodies do not include any sequences that do not have a human origin.

Human antibodies can be prepared by various means. For example, human B cells producing an antigen of interest can be immortalized e.g. by infection with Epstein Barr Virus (EBV). A preferred method for producing human monoclonal antibodies is disclosed in references 3 & 4, in which a human B memory lymphocyte specific for a target antigen is transformed using EBV in the presence of a polyclonal B cell activator. Human monoclonal antibodies can also be produced in non-human hosts by replacing the host's own immune system with a functioning human immune system e.g. into Scid mice or Trimera mice. Mice transgenic for human Ig loci have been successfully used for generating human monoclonal antibodies e.g. the “xeno-mouse” from Abgenix [5]. Phage display has also been successful for generating human antibodies [6], and led to the Humira™ product.

Rather than use human antibodies, the CDR sequences from a non-human antibody can be transferred into a human variable domain in order to create further antibodies sharing their antigen-binding specificity, in the process known as ‘CDR grafting’ [7-12]. The H1, H2 and H3 CDRs may be transferred together into an acceptor V_{H} domain, but it may also be adequate to transfer only one or two of them [10]. Similarly, one two or all three of the L1, L2 and L3 CDRs may be transferred into an acceptor V_{L} domain. Preferred antibodies will have 1, 2, 3, 4, 5 or all 6 of the donor CDRs. Where only one CDR is transferred, it will typically not be the L2 CDR, which is usually the shortest of the six. Typically the donor CDRs will all be from the same antibody, but it is also possible to mix them e.g. to transfer the light chain CDRs from a first antibody and the heavy chain CDRs from a second antibody.

By Kabat numbering [13], the CDRs in a light chain variable region are amino acids 24-34 (L1), 50-56 (L2) & 89-97 (L3), and the CDRs in a heavy chain variable region are amino acids 31-35 (H1), 50-65 (H2) and 95-102 (H3). By Chothia numbering [14], the CDRs in a light chain variable region are amino acids 26-32 (L1), 50-52 (L2) & 91-96 (L3), and the CDRs in a heavy chain variable region are amino acids 26-32 (H1), 53-55 (H2) and 96-101 (H3). Framework residues are variable domain residues other than the CDRs.

As an alternative to CDR grafting, the process of ‘SDR grafting’ may be used [15,16], in which only the specificity-determining residues from within the CDRs are transferred.

The transfer of CDRs or SDRs from a donor variable domain into an acceptor domain may be accompanied by the modification of one or more framework residues, to give a humanised antibody.

Antibodies of the invention may be native antibodies, as naturally found in mammals. Native antibodies are made up of heavy chains and light chains. The heavy and light chains are both divided into variable domains and constant domains. The ability of different antibodies to recognize different antigens arises from differences in their variable domains, in both the light and heavy chains. Light chains of native antibodies in vertebrate species are either kappa (κ) or lambda (λ), based on the amino acid sequences of their constant domains. The constant domain of a native antibody's heavy chains will be α, δ, ε, γ or μ, giving rise respectively to antibodies of IgA, IgD, IgE, IgG, or IgM class. Classes may be further divided into subclasses or isotypes e.g. IgG1, IgG2, IgG3, IgG4, IgA, IgA2, etc. Antibodies may also be classified by allotype e.g. a γ heavy chain may have G1m allotype a, f, x or z, G2m allotype n, or G3m allotype b0, b1, b3, b4, b5, c3, c5, g1, g5, s, t, u, or v; a κ light chain may have a Km(1), Km(2) or Km(3) allotype. A native IgG antibody has two identical light chains (one constant domain C_{L} and one variable domain V_{L}) and two identical heavy chains (three constant domains C_{H}1 C_{H}2 & C_{H}3 and one variable domain V_{H}), held together by disulfide bridges. The domain and three-dimensional structures of the different classes of native antibodies are well known.

Where an antibody of the invention has a light chain with a constant domain, it may be a κ or λ light chain. Where an antibody of the invention has a heavy chain with a constant domain, it may be a α, δ, ε, γ orμ heavy chain. Heavy chains in the γ class (i.e. IgG antibodies) are preferred. The IgG1 subclass is preferred. The Synagis™ antibody is IgG1 with a κ light chain. Antibodies of the invention may have any suitable allotype (see above).

Antibodies of the invention may be fragments of native antibodies that retain antigen binding activity. For instance, papain digestion of native antibodies produces two identical antigen-binding fragments, called “Fab” fragments, each with a single antigen-binding site, and a residual “Fc” fragment without antigen-binding activity. Pepsin treatment yields a “F(ab′)₂” fragment that has two antigen-binding sites. “Fv” is the minimum fragment of a native antibody that contains a complete antigen-binding site, consisting of a dimer of one heavy chain and one light chain variable domain. Thus an antibody of the invention may be Fab, Fab′, F(ab′)₂, Fv, or any other type, of fragment of a native antibody.

An antibody of the invention may be a “single-chain Fv” (“scFv” or “sFv”), comprising a V_{H} and V_{L} domain as a single polypeptide chain [17-19]. Typically the V_{H} and V_{L} domains are joined by a short polypeptide linker (e.g. ≧12 amino acids) between the V_{H} and V_{L} domains that enables the scFv to form the desired structure for antigen binding. A typical way of expressing scFv proteins, at least for initial selection, is in the context of a phage display library or other combinatorial library [20-22]. Multiple scFvs can be linked in a single polypeptide chain [23].

An antibody of the invention may be a “diabody” or “triabody” etc. [24-27], comprising multiple linked Fv (scFv) fragments. By using a linker between the V_{H} and V_{L} domains that is too short to allow them to pair with each other (e.g. <12 amino acids), they are forced instead to pair with the complementary domains of another Fv fragment and thus create two antigen-binding sites.

An antibody of the invention may be a single variable domain or VHH antibody. Antibodies naturally found in camelids (e.g. camels and llamas) and in sharks contain a heavy chain but no light chain. Thus antigen recognition is determined by a single variable domain, unlike a mammalian native antibody [28-30]. The constant domain of such antibodies can be omitted while retaining antigen-binding activity. One way of expressing single variable domain antibodies, at least for initial selection, is in the context of a phage display library or other combinatorial library [31].

An antibody of the invention may be a “domain antibody” (dAb). Such dAbs are based on the variable domains of either a heavy or light chain of a human antibody and have a molecular weight of approximately 13 kDa (less than one-tenth the size of a full antibody). By pairing heavy and light chain dAbs that recognize different targets, antibodies with dual specificity can be made. dAbs are cleared from the body quickly, but can be sustained in circulation by fusion to a second dAb that binds to a blood protein (e.g. to serum albumin), by conjugation to polymers (e.g. to a polyethylene glycol), or by other techniques.

As mentioned above, an antibody of the invention may be a CDR-grafted antibody.

An antibody of the invention may be a chimeric antibody, having constant domains from one organism (e.g. a human) but variable domains from a different organism (e.g. non-human). Chimerisation of antibodies was originally developed in order to facilitate the transfer of antigen specificity from easily-obtained murine monoclonal antibodies into a human antibody, thus avoiding the difficulties of directly generating human monoclonal antibodies.

Thus the term “antibody” as used herein encompasses a range of proteins having diverse structural features (usually including at least one immunoglobulin domain having an all-β protein fold with a 2-layer sandwich of anti-parallel β-strands arranged in two β-sheets), but all of the proteins possess the ability to bind to the pathogen factor.

Antibodies of the invention may include a single antigen-binding site (e.g. as in a Fab fragment or a scFv) or multiple antigen-binding sites (e.g. as in a F(ab′)₂ fragment or a diabody or a native antibody). Where an antibody has more than one antigen-binding site then advantageously it can result in cross-linking of antigens.

Where an antibody has more than one antigen-binding site, the antibody may be mono-specific (i.e. all antigen-binding sites recognize the same antigen) or it may be multi-specific (i.e. the antigen-binding sites recognise more than one antigen). Thus, in a multi-specific antibody, at least one antigen-binding site will recognise a pathogen factor and at least one antigen-binding site will recognise a different antigen.

An antibody of the invention may include a non-protein substance e.g. via covalent conjugation. For example, an antibody may include a radio-isotope e.g. the Zevalin™ and Bexxar™ products include ⁹⁰Y and ¹³¹I isotopes, respectively. As a further example, an antibody may include a cytotoxic molecule e.g. Mylotarg™ is linked to N-acetyl-γ-calicheamicin, a bacterial toxin. As a further example, an antibody may include a covalently-attached polymer, e.g. attachment of polyoxyethylated polyols or polyethylene glycol (PEG), has been reported to increase the circulating half-life of antibodies.

In some embodiments of the invention, an antibody can include one or more constant domains (e.g. including C_{H} or C_{L} domains). As mentioned above, the constant domains may form a κ or λ light chain or an α, δ, ε, γ or μ heavy chain. Where an antibody of the invention includes a constant domain, it may be a native constant domain or a modified constant domain. A heavy chain may include either three (as in α, γ, δ classes) or four (as in μ, ε classes) constant domains. Constant domains are not involved directly in the binding interaction between an antibody and an antigen, but they can provide various effector functions, including but not limited to: participation of the antibody in antibody-dependent cellular cytotoxicity (ADCC); C1q binding; complement dependent cytotoxicity; Fc receptor binding; phagocytosis; and down-regulation of cell surface receptors.

The constant domains can form a “Fc region”, which is the C-terminal region of a native antibody's heavy chain. Where an antibody of the invention includes a Fc region, it may be a native Fc region or a modified Fc region. A Fc region is important for some antibodies' functions e.g. the activity of Herceptin™ is Fc-dependent. Although the boundaries of the Fc region of a native antibody may vary, the human IgG heavy chain Fc region is usually defined to stretch from an amino acid residue at position Cys226 or Pro230 to the heavy chain's C-terminus. The Fc region will typically be able to bind one or more Fc receptors, such as a FcγRI (CD64), a FcγRII (e.g. FcγRIIA, FcγRIIB1, FcγRIIB2, FcγRIIC), a FcγRIII (e.g. FcγRIIIA, FcγRIIIB), a FcRn, FcαR (CD89), FcδR, FcμR, a FcεRI (e.g. FcεRIαβγ₂ or FcεRIαγ₂), FcεRII (e.g. FcεRIIA or FcεRIIB), etc. The Fc region may also or alternatively be able to bind to a complement protein, such as C1q. Modifications to an antibody's Fc region can be used to change its effector function(s) e.g. to increase or decrease receptor binding affinity. For instance, reference 32 reports that effector functions may be modified by mutating Fc region residues 234, 235, 236, 237, 297, 318, 320 and/or 322. Similarly, reference 33 reports that effector functions of a human IgG1 can be improved by mutating Fc region residues (EU Index Kabat numbering) 238, 239, 248, 249, 252, 254, 255, 256, 258, 265, 267, 268, 269, 270, 272, 276, 278, 280, 283, 285, 286, 289, 290, 292, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 and/or 439. Modification of Fc residues 322, 329 and/or 331 is reported in reference 34 for modifying C1q affinity of human IgG antibodies, and residues 270, 322, 326, 327, 329, 331, 333 and/or 334 are selected for modification in reference 35. Mapping of residues important for human IgG binding to FcRI, FcRII, FcRIII, and FcRn receptors is reported in reference 36, together with the design of variants with improved FcR-binding properties. Mutation of the Fc region of available monoclonal antibodies to vary their effector functions is known e.g. reference 37 reports mutation studies for RITUXAN™ to change C1q-binding, and reference 38 reports mutation studies for NUMAX™ to change FcR-binding, with mutation of residues 252, 254 and 256 giving a 10-fold increase in FcRn-binding without affecting antigen-binding.

Antibodies of the invention will typically be glycosylated. N-linked glycans attached to the C_{H}2 domain of a heavy chain, for instance, can influence C1q and FcR binding [36], with aglycosylated antibodies having lower affinity for these receptors. The glycan structure can also affect activity e.g. differences in complement-mediated cell death may be seen depending on the number of galactose sugars (0, 1 or 2) at the terminus of a glycan's biantennary chain. An antibody's glycans preferably do not lead to a human immunogenic response after administration.

Antibodies of the invention can be prepared in a form free from products with which they would naturally be associated. Contaminant components of an antibody's natural environment include materials such as enzymes, hormones, or other host cell proteins.

Antibodies of the invention can be used directly (e.g. as the active ingredient for pharmaceuticals or diagnostic reagents), or they can be used as the basis for further development work. For instance, an antibody may be subjected to sequence alterations or chemical modifications in order to improve a desired characteristic e.g. binding affinity or avidity, pharmacokinetic properties (such as in vivo half-life), etc. Techniques for modifying antibodies in this way are known in the art. For instance, an antibody may be subjected to “affinity maturation”, in which one or more residues (usually in a CDR) is mutated to improve its affinity for a target antigen. Random or directed mutagenesis can be used, but reference 39 describes affinity maturation by V_{H} and V_{L} domain shuffling as an alternative to random point mutation. Reference 40 reports how NUMAX™ was derived by a process of in vitro affinity maturation of the CDRs of the heavy and light chains of SYNAGIS™, giving an antibody with enhanced potency and 70-fold greater binding affinity for RSV F protein.

Preferred antibodies of the invention are specific for one of the pathogen factors described below. Thus the antibody will have a tighter binding affinity for that antigen than for an arbitrary control antigen e.g. than for a human protein. Preferred antibodies have nanomolar or picomolar affinity constants for target antigens e.g. 10⁻⁹ M, 10⁻¹⁰ M, 10⁻¹¹ M, 10⁻¹² M, 10⁻¹³ M or tighter).

The term “monoclonal” as originally used in relation to antibodies referred to antibodies produced by a single clonal line of immune cells, as opposed to “polyclonal” antibodies that, while all recognizing the same target protein, were produced by different B cells and would be directed to different epitopes on that protein. As used herein, the word “monoclonal” does not imply any particular cellular origin, but refers to any population of antibodies that all have the same amino acid sequence and recognize the same epitope in the same target protein. Thus a monoclonal antibody may be produced using any suitable protein synthesis system, including immune cells, non-immune cells, acellular systems, etc. This usage is usual in the field: the product datasheets for the CDR-grafted humanised antibody Synagis™ expressed in a murine myeloma NSO cell line, the humanised antibody Herceptin™ expressed in a CHO cell line, and the phage-displayed antibody Humira™ expressed in a CHO cell line all refer the products as monoclonal antibodies.

### Antibody-Based Pharmaceutical Compositions

The use of antibodies as the active ingredient of pharmaceuticals is now widespread, including products such as Herceptin™ (trastuzumab) and Synagis™ (palivizumab). Synagis™ and Numax™ (motavizumab) in particular are effective in preventing pathogen-caused disease. The invention thus provides a pharmaceutical composition containing one or more antibody(ies) of the invention. Techniques for purification of monoclonal antibodies to a pharmaceutical grade are well known in the art.

A pharmaceutical composition will usually contain one or more pharmaceutically acceptable carriers and/or excipient(s). A thorough discussion of such components is available in reference 41. These may include liquids such as water, saline, glycerol and ethanol. Additionally, auxiliary substances, such as wetting or emulsifying agents or pH buffering substances, may be present in such compositions.

Pharmaceutical compositions may be prepared in various forms e.g. as injectables, either as liquid solutions or suspensions. Solid forms suitable for solution in, or suspension in, liquid vehicles prior to injection can also be prepared (e.g. a lyophilised composition, like Synagis™ and Herceptin™, for reconstitution with sterile water or buffer, optionally containing a preservative). The composition may be prepared for topical administration e.g. as an ointment, cream or powder. The composition may be prepared for oral administration e.g. as a tablet or capsule, as a spray, or as a syrup (optionally flavoured), in which case it will usually contain agents to protect the active ingredients from degradation. The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted (e.g. with sterile water or a sterile buffer) at the time of use, prior to administration to a patient e.g. an antibody can be provided in dry form.

Preferred pharmaceutical forms for administration of antibodies include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain carriers/excipients such as suspending, preservative, stabilising and/or dispersing agents.

Pharmaceutical compositions will generally have a pH between 5.5 and 8.5, preferably between 6 and 8, and more preferably about 7. The pH may be maintained by a buffer.

The composition will usually be sterile. The composition will usually be non-pyrogenic e.g. containing <1 EU (endotoxin unit, a standard measure) per dose, and preferably <0.1 EU per dose.

The composition is preferably gluten free. The composition may be substantially isotonic with respect to humans.

Compositions may include an antimicrobial and/or preservative.

Compositions may comprise a detergent. Where present, detergents are generally used at low levels e.g. <0.01%.

Compositions may include sodium salts (e.g. sodium chloride) to give tonicity. A concentration of 10±2 mg/ml NaCl is typical.

Compositions may comprise a sugar alcohol (e.g. mannitol) or a disaccharide (e.g. sucrose or trehalose) e.g. at around 15-30mg/ml (e.g. 25 mg/ml), particularly if they are to be lyophilised or if they include material which has been reconstituted from lyophilised material.

Compositions may include free amino acids e.g. histidine. For instance, reference 42 discloses an improved aqueous formulation for the Synagis™ antibody comprising histidine in an aqueous carrier.

Pharmaceutical compositions will include an effective amount of the active ingredient. The concentration of the ingredient in a composition will, of course, vary according to the volume of the composition o be delivered, and known antibody-based pharmaceuticals provide guidance in this respect. For example, Synagis™ is provided for reconstitution to give 50 mg antibody in 0.5 ml or 100 mg of antibody in 1.0 ml. The appropriate volume is delivered to a patient based on their recommended dose.

Once formulated, the compositions of the invention can be administered directly to the subject (see below). It is preferred that the compositions are adapted for administration to human subjects. This will generally be in liquid (e.g. aqueous) form.

In compositions that include antibodies, particularly pharmaceutical compositions, the antibodies preferably make up at least 50% by weight (e.g. at least 60%, 70%, 80%, 90%, 95%, 97%, 98%, 99% or more) of the total protein in the composition. The antibodies are thus in purified form.

Pharmaceutical compositions of the invention are preferably supplied in hermetically-sealed containers.

The invention also provides a method of preparing a pharmaceutical composition, comprising a step of admixing an antibody of the invention with one or more pharmaceutically acceptable ingredients.

### Non-Antibody Antagonists

As well as using antibodies to prevent the interaction between Factor H and a pathogen factor, non-antibody active ingredients may also be used. Such non-antibody molecules may be identified using suitable screening assays. For instance, an assay may involve incubating a Factor H protein, a pathogen factor and a candidate compound under conditions where the Factor H and pathogen factor would normally be able to interact. If the presence of the candidate compound inhibits that interaction then the candidate compound may be suitable for use with the invention.

The screening assay can take various forms. For instance, the Factor H protein and candidate compound may be mixed with each other, and then mixed with the pathogen factor. As an alternative, the pathogen factor and candidate compound may be mixed with each other, and then mixed with the Factor H protein. As a further alternative, all three components may be mixed together. As a further alternative the Factor H protein and pathogen factor may be mixed and permitted to interact, and the ability of a candidate compound to disrupt the interaction may be assayed. In all cases, however, the assay aims to identify whether a compound can inhibit the natural interaction between the pathogen factor and Factor H.

Typical candidate compounds that can be assessed include, but are not restricted to, peptides, peptoids, proteins, lipids, metals, small organic molecules, RNA aptamers, antibiotics and other known pharmaceuticals, polyamines, and combinations or derivatives thereof. Small organic molecules have a molecular weight of about more than 50 and less than about 2,500 daltons, and most preferably between about 300 and about 800 daltons. Candidate compounds may be derived from large libraries of synthetic or natural compounds. For instance, synthetic compound libraries are commercially available from many commercial suppliers. Libraries of natural compounds in the form of bacterial, fungal, plant and animal extracts may also be used. Candidate compounds may be synthetically produced using combinatorial chemistry either as individual compounds or as mixtures.

Factor H Supplements and Decoys

Another way of preventing a pathogen factor from sequestering available Factor H is to provide a patient with extra Factor H, thereby replacing the function of sequestered factor H, or to provide a Factor H decoy, thereby preventing factor H from being sequestered.

For example, a patient with a hemorrhagic syndrome (or at risk of developing one) due to the lack of functional fH could receive fH supplements. Similarly, a patient with a hemorrhagic syndrome (e.g. at an early stage) or at risk of developing one could receive a fH decoy in order to prevent their endogenous fH from being sequestered by a pathogen factor.

Factor H for supplementation can be prepared in various ways. For instance, factor H can be purified from plasma or blood. Rather than use blood-derived proteins, however, it is safer to use recombinant factor H. Methods for recombinant expression of factor H proteins are known in the art e.g. including expression in yeast cells [43] and insect cells [44, 45].

Supplemented factor H may have a wild-type sequence or may be a factor H analog that provides the natural function of factor H, in particular its effects on C3b (binding to C3b and acting as a cofactor for serine esterase factor I, resulting in C3b cleavage to form iC3b). Such analogs can include non-human forms of factor H, or modified forms such as those having fH's complement control protein modules 1-4, 1-5 or 1-6 [43]. The aim is to replace factor H function that has been lost by pathogen sequestration. Wild-type fH sequences include SEQ ID NO: 9 (isoform a) and SEQ ID NO: 10 (isoforms b, also known as FHL-1).

Factor H decoys should have a higher affinity for the pathogen factor than endogenous factor H. Such decoys can be prepared by, for instance, mutagenesis or in vitro evolution of wild-type factor H, followed by a binding assay using a pathogen factor of interest. The affinity for pathogen factors of natural factor H mutants and isoforms can also be tested in this way, as can analogs of factor H and non-human forms of factor H. The binding assay can rapidly reveal the decoy's binding affinity relative to wild-type factor H (e.g. relative to the mature form of SEQ ID NO: 9 i.e. residues 19-1231). The decoy may or may not retain factor H's natural complement functions. For instance, reference 44 reports C-terminus truncated mutants with modified complement regulatory functions.

Decoys without natural complement functions may be used as factor H antagonists (see above).

Known factor H mutants include, but are not limited to: E1172Stop, R1210C, and R1215G [45]; W1183L, V1197A, or R1210C [46]; and I62V or Y402H [47].

### Active Immunisation

The invention provides an immunisation method for preventing or treating a hemorrhagic syndrome caused by a pathogen. A patient is immunised with a protein that shares an epitope with a pathogen factor that can bind to fH. The resulting immune response can block the ability of the pathogen factor to sequester fH.

Where the pathogen is a meningococcus, the patient is immunised with a protein that shares one or more epitopes with a meningococcal NMB1870 protein. NMB1870 was originally disclosed as protein ‘741’ from serogroup B strain MC58 [SEQ IDs 2535 & 2536 in ref. 48]. It has also been referred to as ‘GNA1870’ [refs. 49-51], ‘ORF2086’ [52-55] and FHBP [56,57]. Its 3D solution structure is reported in reference 58. Sequences for numerous strains are reported in reference 59, where 56% of amino acids were shown to be conserved in all isolates. This lipoprotein is expressed across all meningococcal serogroups and has been found in multiple meningococcal strains. NMB1870 sequences have been grouped into three main families [49], and the invention may use a NMB18710 from 1, 2 or 3 of these families. Prototype sequences for each family are given herein as SEQ ID NOS: 1 to 3. Immunisation with NMB1870 to provide an anti-NMB1870 antibody response has been reported many times. Identification of immunologically-active fragments of the full-length protein has also been reported.

Suitable forms of NMB1870 for use in immunisation include lipoproteins e.g. expressed in E. coli. For instance, the lipoprotein may have a N-terminal cysteine residue, to which a lipid is covalently attached. Any of the proteins disclosed in references 52 & 53 may be used. Other forms of NMB1870 include fusion proteins e.g. fused to NMB2091 (e.g. SEQ ID NO: 8 of reference 60), or fused to variants of NMB1870 (e.g. see ref. 61).

Whereas previous studies of immunisation with NMB1870 focused on the protein's ability of elicit bactericidal antibodies, the present invention is interested more in antibodies that can prevent interaction with fH than with antibodies that are themselves bactericidal. Antibodies according to the invention may also be bactericidal, but the main concern is blocking fH binding.

Where the pathogen is a West Nile virus, the patient is immunised with a protein that shares one or more epitopes with a West Nile virus NS1 non-structural protein. The NS1 protein is a proteolytic product of the full-length viral polyprotein, and its REFSEQ sequence (GI:27735303) is SEQ ID NO: 4 herein. Immunisation with WNV NS1 to provide an anti-NS1 antibody response has been reported many times, and epitope mapping has also been performed e.g. see FIG. 6 of reference 62.

Where the pathogen is a Dengue virus, the patient is immunised with a protein that shares one or more epitopes with a Dengue virus NS1 non-structural protein. The NS1 protein is a proteolytic product of the full-length viral polyprotein. The invention may use NS1 epitope(s) from 1, 2, 3 or 4 of Dengue virus types 1, 2, 3 and/or 4. Prototypic NS1 sequences for each of the four virus types, respectively, are SEQ ID NOS: 5 to 8 herein. Immunisation with Dengue virus NS1 proteins to provide an anti-NS1 antibody response has been reported many times, and epitope mapping has also been performed e.g. see refs 63 & 64.

Thus the invention may involve immunising a patient with a polypeptide comprising an amino acid sequence that:
(i) is at least i % identical to an amino acid sequence selected from the group consisting of SEQ ID NOS: 1, 2, 3, 4, 5, 6, 7, 8, 11; and/or
(ii) is a fragment of at least j contiguous amino acids of an amino acid sequence selected from the group consisting of SEQ ID NOS: 1, 2, 3, 4, 5, 6, 7, 8, 11; and/or
(iii) has 1, 2, 3, 4, 5, 6, 7, 8, 9, 10, 11, 12, 13, 14, 15, 16, 17, 18, 19 or 20 single amino acid alterations (deletions, insertions, substitutions), which may be at separate locations or may be contiguous, relative to an amino acid sequence selected from the group consisting of SEQ ID NOS: 1, 2, 3, 4, 5, 6, 7, 8, 11;

The value of i is at least 50 e.g. 50, 60, 70, 80, 85, 90, 95, 96, 97, 98, 99 or 100. The value of j is at least 7 e.g. 7, 8, 9, 10, 11, 12, 13, 14, 15, 16, 17, 18, 19, 20, 22, 24, 26, 28, 30, 35, 40, 45, 50 or more.

The administered polypeptide will elicit an immune response that recognises the natural pathogen factor e.g. recognises a polypeptide consisting of SEQ ID NO: 1, 2, 3, 4, 5, 6, 7, 8 or 11.

As an alternative to using polypeptides to immunise patients, nucleic acid (preferably DNA e.g. in the form of a plasmid) encoding the polypeptide may be used. For example, DNA immunisation using Dengue virus NS1 sequences has been reported [65].

### Pathogen-Induced Hemorrhagic Syndromes

In some embodiments, the invention relates to the prevention and/or treatment of various pathogen-induced hemorrhagic syndromes.

As well as causing meningitis, meningococcus can cause hemorrhagic symptoms, particularly in association with fulminant meningococcemia. Meningococcal infection can also lead to Waterhouse-Friderichsen Syndrome, meningococcal hemorrhagic adrenalitis or purpura fulminans.

As well as causing dengue, dengue virus can cause dengue hemorrhagic fever (DHF). Dengue and DHF can be caused by any of dengue virus types 1, 2, 3 and 4. Dengue symptoms include high fever, severe headache, backache, joint pains, nausea, vomiting, eye pain and rashes. In DHF, fever lasts from 2-7 days with normal dengue symptoms, but is followed by hemorrhagic manifestations, a tendency to bruise easily, skin hemorrhages (petechiae and/or ecchymoses), bleeding nose and/or gums, and sometimes internal bleeding. DHF is normally treated by fluid replacement therapy. DHF is also known as hemorrhagic dengue, dengue shock syndrome, Philippine hemorrhagic fever, Thai hemorrhagic fever or Singapore hemorrhagic fever.

As well as causing West Nile fever and West Nile encephalomyelitis, West Nile virus can cause a hemorrhagic fever [1].

Neisseria gonorrhoeae (gonococcus) can cause a hemorrhagic conjunctivitis. This condition may be prevented and/or treated according to the invention e.g. using the gonococcal homolog of NMB1870 (NGO0033; SEQ ID NO: 11 herein).

### Complement and Patient Groups

NMB1870 is known for use in immunising against meningococcal infection. If its interaction with fH leads to hemorrhagic symptoms by interfering with natural regulation of C3 complement then such immunisation will be particularly suited for patients having a functional C3 component. In contrast, patients with a deficient C3 component can be expected to require immunisation with at least one further immunogen in addition to NMB1870. Such further immunogens may be selected from, for instance: NMB2132; NadA; meningococcal lipooligosaccharide: TbpA; TbpB; NhhA; NspA; Omp85; PorA; outer membrane vesicles: etc.

Similarly, immunisation with West Nile virus NS1 protein will be particularly suited for patients having a functional C3 component, but patients with a deficient C3 component may require immunisation with at least one further immunogen e.g. selected from: an envelope protein; a capsid protein; a NS2a protein; a NS2b protein; a NS3 protein; a NS4a protein; a NS4b protein; a NS5 protein; a protease; etc.

Similarly, immunisation with dengue virus NS1 protein will be particularly suited for patients having a functional C3 component, but patients with a deficient C3 component may require immunisation with at least one further immunogen e.g. selected from: an envelope protein; a capsid protein; a NS2a protein; a NS2b protein; a NS3 protein; a NS4a protein; a NS4b protein; a NS5 protein; a protease; etc.

Where the additional meningococcal or viral antigen is a polypeptide, it may be one of the polypeptides listed, a homolog of one of the polypeptides, a fusion protein comprising one of the polypeptides, a protein comprising an epitope from one of the polypeptides, etc.

Suitable forms of NMB1870 for use in immunisation include those described above.

### Medical Treatments and Uses

Antibodies of the invention may be used for the treatment and/or prevention of hemorrhagic diseases, particularly in humans. Thus the invention provides an antibody of the invention for use in therapy e.g. in preventing and/or treating a hemorrhagic disease. Also provided is a method of treating a patient comprising administering to that patient an antibody of the invention. Also provided is the use of an antibody of the invention, in the manufacture of a medicament for the treatment and/or prevention of a hemorrhagic disease.

To confirm efficacy after administration of an antibody composition of the invention, any known methods for assessing the presence and/or severity of hemorrhagic symptoms can be used.

Treatment may be targeted at patient groups that are particularly at risk of or susceptible to hemorrhagic syndromes.

Pharmaceutical compositions of the invention may be administered by any number of routes including, but not limited to, intravenous, intramuscular, intra-arterial, intramedullary, intraperitoneal, intrathecal, intraventricular, transdermal, transcutaneous, oral, topical, subcutaneous, intranasal, enteral, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule. Known antibody-based pharmaceuticals provide some guidance relating to frequency of administration e.g. whether a pharmaceutical should be delivered daily, weekly, monthly, etc. Frequency and dosage may also depend on the severity of symptoms.

Patients will receive an effective amount of the active ingredient i.e. an amount that is sufficient to treat, ameliorate, or prevent a hemorrhagic syndrome. Therapeutic effects may also include reduction in physical symptoms. The optimum effective amount for any particular subject will depend upon their size and health, the nature and extent of the condition, and the therapeutics or combination of therapeutics selected for administration. The effective amount delivered for a given situation can be determined by routine experimentation and is within the judgment of a clinician. For purposes of the present invention, an effective dose will generally be from about 0.01 mg/kg to about 50 mg/kg, or about 0.05 mg/kg to about 10 mg/kg of the compositions of the present invention in the individual to which it is administered. Known antibody-based pharmaceuticals provide guidance in this respect e.g. Herceptin™ is administered by intravenous infusion of a 21 mg/ml solution, with an initial loading dose of 4 mg/kg body weight and a weekly maintenance dose of 2 mg/kg body weight; Rituxan™ is administered weekly at 375 mg/m²; Synagis™ is administered intramuscularly at 15 mg/kg body weight, typically once a month during the RSV season; etc.

Antibodies of the invention may be administered (either combined or separately) with other therapeutics e.g. with fluid replacement therapy, with anti-inflammatories, etc.

### Nucleic Acids and Recombinant Antibody Expression

The invention encompasses nucleic acid sequences encoding antibodies of the invention. Where an antibody of the invention has more than one chain (e.g. a heavy chain and a light chain), the invention encompasses nucleic acids encoding each chain. The invention also encompasses nucleic acid sequences encoding the amino acid sequences of CDRs of antibodies of the invention.

Nucleic acids encoding the antibodies can be prepared from cells, viruses or phages that express an antibody of interest. For instance, nucleic acid can be prepared from an immortalised B cell of interest, and the gene(s) encoding the antibody of interest can then be cloned and used for subsequent recombinant expression. Expression from recombinant sources is more common for pharmaceutical purposes than expression from B cells or hybridomas e.g. for reasons of stability, reproducibility, culture ease, etc. Methods for obtaining and sequencing immunoglobulin genes from B cells are well known in the art e.g. see reference 66. Thus various steps of culturing, sub-culturing, cloning, sub-cloning, sequencing, nucleic acid preparation, etc. can be performed in order to perpetuate the antibody expressed by a cell or phage of interest. The invention encompasses all cells, nucleic acids, vectors, sequences, antibodies etc. used and prepared during such steps.

The invention provides a method for preparing one or more nucleic acid molecules (e.g. heavy and light chain genes) that encodes an antibody of interest, comprising the steps of: (i) providing an immortalised B cell clone expressing an antibody of interest; (ii) obtaining from the B cell clone nucleic acid that encodes the antibody of interest. The nucleic acid obtained in step (ii) may be inserted into a different cell type, or it may be sequenced.

The invention also provides a method for preparing a recombinant cell, comprising the steps of: (i) obtaining one or more nucleic acids (e.g. heavy and/or light chain genes) from a B cell clone that encodes an antibody of interest; and (ii) inserting the nucleic acid into an expression host in order to permit expression of the antibody of interest in that host.

Similarly, the invention provides a method for preparing a recombinant cell, comprising the steps of: (i) sequencing nucleic acid(s) from a B cell clone that encodes the antibody of interest; and (ii) using the sequence information from step (i) to prepare nucleic acid(s) for inserting into an expression host in order to permit expression of the antibody of interest in that host.

Recombinant cells produced in these ways can then be used for expression and culture purposes. They are particularly useful for expression of antibodies for large-scale pharmaceutical production.

The invention provides a method for preparing an antibody of the invention, comprising a step of culturing a cell such that it produces the antibody. The methods may further comprise a step of recovering the antibody that has been produced, to provide a purified antibody. A cell used in these methods may, as described elsewhere herein, be a recombinant cell, an immortalised B cell, or any other suitable cell. Purified antibody from these methods can then be used in pharmaceutical and/or diagnostic compositions, etc.

Cells for recombinant expression include bacteria, yeast and animal cells, particularly mammalian cells (e.g. CHO cells, human cells such as PER.C6 (ECACC deposit 96022940 [67]) or HKB-11 [68,69] cells), etc.), as well as plant cells. Preferred expression hosts can glycosylate the antibody of the invention, particularly with carbohydrate structures that are not themselves immunogenic in humans (see above). Expression hosts that can grow in serum-free media are preferred. Expression hosts that can grow in culture without the presence of animal-derived products are preferred.

The expression host may be cultured to give a cell line.

Nucleic acids used with the invention may be manipulated to insert, delete or amend certain nucleic acid sequences. Changes from such manipulation include, but are not limited to, changes to introduce restriction sites, to amend codon usage, to add or optimise transcription and/or translation regulatory sequences, etc. It is also possible to change the nucleic acid to alter the encoded amino acids. For example, it may be useful to introduce one or more (e.g. 1, 2, 3, 4, 5, 6, 7, 8, 9, 10, etc.) amino acid substitutions, one or more (e.g. 1, 2, 3, 4, 5, 6, 7, 8, 9, 10, etc.) amino acid deletions and/or one or more (e.g. 1, 2, 3, 4, 5, 6, 7, 8, 9, 10, etc.) amino acid insertions into the antibody's amino acid sequence. Such point mutations can modify effector functions, antigen-binding affinity, post-translational modifications, immunogenicity, etc., can introduce amino acids for the attachment of covalent groups (e.g. labels) or can introduce tags (e.g. for purification purposes). Mutations can be introduced in specific sites or can be introduced randomly, followed by selection (e.g. molecular evolution).

Nucleic acids of the invention may be present in a vector (such as a plasmid) e.g. in a cloning vector or in an expression vector. Thus a sequence encoding an amino acid sequence of interest may be downstream of a promoter such that its transcription is suitable controlled. The invention provides such vectors, and also provides cells containing them.

The invention also provides an immortalised human B cell that can secrete an antibody of the invention.

### Animal Models

NMB1870 binds to human fH but not to mouse or rat fH. To facilitate animal studies of NMB1870 and other pathogen factors, the invention provides a non-human mammal (e.g. a rodent, a mouse, a rat, a guinea pig, a hamster, a rabbit, a goat, etc.) that expresses a human factor H protein. Such animals may be made by standard transgenic approaches eg. homologous recombination or gene targeting to replace the animal's natural factor H with a human sequence. Mutagenesis of the animal's own fH sequence to give it human characteristics is also a possibility.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necesary, the word “substantially” may be omitted from the definition of the invention.

The term “about” in relation to a numerical value x means, for example, x±10%.

Different steps in a method of the invention can optionally be performed at different times by different people in different places (e.g. in different countries).

### MODES FOR CARRYING OUT THE INVENTION

NMB1870 binds to human fH but not to mouse or rat fH. A transgenic mouse is prepared in which the native murine factor H gene has been replaced by a human factor H gene. The mouse develops normally, but expresses human factor H that can bind to NMB1870. When NMB1870 is injected into these mice, some of them may develop hemorrhagic symptoms.

Human anti-NMB1870 antibodies are prepared using the EBV transformation methods disclosed in reference 4. These are screened to find antibodies that can inhibit the ability of NMB1870 to bind to human factor H. These inhibitory antibodies are then co-administered to mice with NMB1870 to inhibit any hemorrhagic symptoms.

### REFERENCES

### The Contents of Which are Hereby Incorporated by Reference

[1] Paddock et al. (2006) Clin Infect Dis 42:1527-35.
[2] Chung et al. (2006) PNAS 103:19111-6.
[3] WO2004/076677.
[4] Traggiai et al. (2004) Nat Med. 10(8):871-5.
[5] Green (1999) J Immunol Methods. 231(1-2):11-23.
[6] Mancini et al. (2004) New Microbiol. 27(4):315-28.
[7] Ewert et al. (2004) Methods 34(2):184-99.
[8] Riechmann et al. (1988) Nature 332:323-327.
[9] O'Brien & Jones (2003) Methods Mol Biol. 207:81-100.
[10] Iwahashi et al. (1999) Mol Immunol. 36(15-16):1079-91.
[11] Lo (2004) Methods Mol Biol. 248:135-59.
[12] Verhoeyen et al. (1988) Science 239: 1534-1536.
[13] Kabat et al. (1991) Sequences of Proteins of Immunological Interest, 5th Ed. Public Health Service, National Institutes of Health, Bethesda. Md.
[14] Chothia & Lesk (1987) Mol. Biol. 196:901-917.
[15] Kashmiri et al. (2005) Methods 36(1):25-34.
[16] Gonzales et al. (2004) Mol Immunol. 41(9):863-72.
[17] Worn & Pluckthun (2001) J Mol Biol. 305(5):989-1010.
[18] WO93/16185
[19] Adams & Schier (1999) J Immunol Methods. 231(1-2):249-60.
[20] Hallborn & Carlsson (2002) Biotechniques Suppl:30-7.
[21] Pini & Bracci (2000) Curr Protein Pept Sci 1(2):155-69.
[22] Walter et al. (2001) Comb Chem High Throughput Screen. 4(2):193-205.
[23] Gruber et al. (1994) J Immunol 152(11):5368-74.
[24] U.S. Pat. No. 5,591,828
[25] WO 93/11161.
[26] Hollinger et al. (1993) Proc. Natl. Acad. Sci. USA 90:6444-6448.
[27] Hudson & Kortt (1999) J Immunol Methods 231:177-89.
[28] Muyldermans (2001) J Biotechnol 74(4):277-302.
[29] Dumoulin et al. (2002) Protein Sci. 11(3):500-15.
[30] Sidhu et al. (2004) J Mol Biol. 338(2):299-310.
[31] Kotz et al. (2004) Eur J Biochem. 271(9): 1623-9.
[32] U.S. Pat. No. 5,624,821.
[33] U.S. Pat. No. 6,737,056.
[34] U.S. Pat. No. 6,538,124.
[35] U.S. Pat. No. 6,528,624.
[36] Shields et al. (2001) J Biol Chem 276:6591-604.
[37] Idusogie et al. (2000) J Immunol 164(8):4178-84.
[38] Dall'acqua et al. (2006) J Biol Chem 281(33):23514-24.
[39] Marks et al. (1992) Bio/Technology 10:779-83.
[40] Wu et al. (2005) J Mol Biol 350(1):126-44.
[41] Gennaro (2000) Remington: The Science and Practice of Pharmacy, 20th edition, ISBN: 0683306472.
[42] U.S. Pat. No. 7,132,100.
[43] US-2006/0178308.
[44] Pangburn (2002) J Immunol 169:4702-6.
[45] Manuelian et al. (2003) J Clin Invest. 111(8): 1181-1190.
[46] Sánchez-Corral et al. (2002) American Journal of Human Genetics 71:1285-1295.
[47] US-2007/0020647.
[48] WO99/57280.
[49] Masignani et al. (2003) J Exp Med 197:789-799.
[50] Welsch et al. (2004) J Immunol 172:5606-15.
[51] Hou et al. (2005) J Infect Dis 192(4):580-90.
[52] WO03/063766.
[53] WO2004/094596.
[54] Fletcher et al. (2004) Infect Immun 72:2088-2100.
[55] Zhu et al. (2005) Infect Immun 73(10):6838-45.
[56] Madico et al. (2006) J Immunol 177:501-10.
[57] Beernink et al. (2006) Clin Vaccine Immunol. 13(7):758-63.
[58] Cantini et al. (2006) J Biol Chem 281:7220-7.
[59] Jacobsson et al. (2006) Vaccine 24:2161-8.
[60] WO2004/032958.
[61] PCT/IB2006/003876.
[62] Chung et al. (2006) J Virol. 80(3):1340-51.
[63] Wu et al. (2001) J Clin Microbiol. 39(3):977-82.
[64] Huang et al. (1999) J Med Virol. 57(1):1-8.
[65] Costa et al. (2007) Virology 358:413-23.
[66] Chapter 4 of Kuby Immunology (4th edition, 2000; ASIN: 0716733315
[67] Jones et al. Biotechnol Prog 2003, 19(1):163-8
[68] Cho et al. Cytotechnology 2001, 37:23-30
[69] Cho et al. Biotechnol Prog 2003, 19:229-32

1. A method for treating a hemorrhagic syndrome caused by a pathogen in a patient, comprising a step of administering to the patient a medicament that prevents the interaction between Factor H and a pathogen factor.

2. The method of claim 1, wherein the medicament has an antibody as an active ingredient.

3. The method of claim 2, wherein the pathogen is Neisseria meningitidis and the antibody recognises NMB1870.

4. The method of claim 2, wherein the pathogen is West Nile virus and the antibody recognises West Nile virus NS1 protein.

5. The method of claim 2, wherein the pathogen is dengue virus and the antibody recognises dengue virus NS1 protein.

6. The method of any one of claims 2 to 5, wherein the antibody is a CDR-grafted, humanised or human antibody.

7. A non-murine monoclonal antibody that binds to a pathogen factor that can bind to Factor H.

8. The antibody of claim 7, wherein the pathogen is Neisseria meningitidis and the pathogen factor is NMB1870.

9. The antibody method of claim 7, wherein the pathogen is West Nile virus and the pathogen factor is West Nile virus NS1 protein.

10. The antibody of claim 7, wherein the pathogen is dengue virus and the pathogen factor is dengue virus NS1 protein.

11. The antibody of any one of claims 7 to 10, wherein the antibody is a CDR-grafted, humanised or human antibody.

12. A method for preventing or treating a hemorrhagic syndrome caused by a pathogen, comprising a step of administering to a patient a medicament comprising a protein sharing an epitope with a pathogen factor that can bind to Factor H.

13. The method of claim 12, wherein the pathogen is Neisseria meningitidis and the pathogen factor is NMB1870.

14. The method of claim 12, wherein the pathogen is West Nile virus and the pathogen factor is West Nile virus NS1 protein.

15. The method of claim 12, wherein the pathogen is dengue virus and the pathogen factor is dengue Virus NS1 protein.

16. A method for preventing or treating meningococcal disease in a patient, comprising a step of administering to the patient a NMB1870 protein, wherein the patient has a complement system with a functional C3 component.

17. A method for preventing or treating meningococcal disease in a patient, comprising a step of administering to the patient a NMB1870 protein and at least one other meningococcal immunogen, wherein the patient has a complement system with a deficient C3 component.

18. A method for preventing or treating West Nile virus disease in a patient, comprising a step of administering to the patient a West Nile virus NS1 protein, wherein the patient has a complement system with a functional C3 component.

19. A method for preventing or treating West Nile virus disease in a patient, comprising a step of administering to the patient a West Nile virus NS1 protein and at least one other West Nile virus immunogen, wherein the patient has a complement system with a deficient C3 component.

20. A method for preventing or treating Dengue virus disease in a patient, comprising a step of administering to the patient a Dengue virus NS1 protein, wherein the patient has a complement system with a functional C3 component.

21. A method for preventing or treating Dengue virus disease in a patient, comprising a step of administering to the patient a Dengue virus NS1 protein and at least one other Dengue virus immunogen, wherein the patient has a complement system with a deficient C3 component.


SEQUENCE LISTING (11 sequences)

SEQ ID NO: 1 - protein, 274 aa, Neisseria meningitidis
MNRTAFCCLSLTTALILTACSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 2 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 3 - protein, 281 aa, Neisseria meningitidis
MNRTAFCCLSLTTALILTACSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 4 - protein, 352 aa, West Nile Virus
DTGCAIDIGRQELRCGSGVFIHNDVEAWMDRYKFYPETPQGLAKIIQKAHAEGVCGLRSVSRLEHQMWEAIKDELNTLLKENGVDLSVVVEKQNGMYKAAPKRLAATTEKLEMGWKAWGKSIIFAPELANNTFVIDGPETEECPTANRAWNSMEVEDFGFGLTSTRMFLRIRETNTTECDSKIIGTAVKNNMAVHSDLSYWIESGLNDTWKLERAVLGEVKSCTWPETHTLWGDGVLESDLIIPITLAGPRSNHNRRPGYKTQNQGPWDEGRVEIDFDYCPGTTVTISDSCEHRGPAARTTTESGKLITDWCCRSCTLPPLRFQTENGCWYGMEIRPTRHDEKTLVQSRVNA

SEQ ID NO: 5 - protein, 352 aa, Dengue virus type 1
DSGCVINWKGRELKCGSGIFVTNEVHTWTEQYKFQADSPKRLSAAIGKAWEEGVCGIRSATRLENIMWKQISNELNHILLENDMKLTVVVGDVTGILAQGKKMIRPQPMEHKYSWKSWGKAKITGADVQNTTFIIDGPNTPECPDNQRAWNIWEVEDYGFGVFTTNIWLKLRDSYTQVCDHRLMSAAIKDSKAVHADMGYWIESEKNETWKLERASFIEVKTCIWPRSHTLWSNGVLESEMIIPKIYGGPVSQHNYRPGYFTQTAGPWHLGKLELDFDLCEGTTVVVDENCGNRGPSLRTTTVTGKTIHEWCCRSCTLPPLRFKGEDGCWYGMEIRPVKEKEENLVKSMVSA

SEQ ID NO: 6 - protein, 352 aa, Dengue virus type 2
DSGCVVSWKNKELKCGSGIFVTDNVHTWTEQYKFQPESPSKLASAIQKAHEEGICGIRSVTRLENLMWKQITSELNHILSENEVKLTIMTGDIKGIMQVGKRSLRPQPTELRYSWKTWGKAKMLSTELHNQTFLIDGPETAECPNTNRAWNSLEVEDYGFGVFTTNIWLRLREKQDAFCDSKLMSAAIKDNRAVHADMGYWIESALNDTWKIEKASFIEVKSCHWPKSHTLWSNGVLESEMVIPKNFAGPVSQHNNRPGYHTQTAGPWHLGKLEMDFDFCEGTTVVVTEDCGNRGPSLRTTTASGKLITEWCCRSCTLPPLRYRGEDGCWYGMEIRPLKEKEENLVSSLVTA

SEQ ID NO: 7 - protein, 352 aa, Dengue virus type 3
DMGCVINWKGKELKCGSGIFVTNEVHTWTEQYKFQADSPKRLATAIAGAWENGVCGIRSTTRMENLLWKQIANELNYILWENNIKLTVVVGDIIGVLEQGKRTLTPQPMELKYSWKTWGKAKIVTAETQNSSFIIDGPNTPECPSASRAWNVWEVEDYGFGVFTTNIWLKLREMYTQLCDHRLMSAAVKDERAVHADMGYWIESQKNGSWKLEKASLIEVKTCTWPKSHTLWSNGVLESDMIIPKSLAGPISQHNYRPGYHTQTAGPWHLGKLELDFNYCEGTTVVITENCGTRGPSLRTTTVSGKLIHEWCCRSCTLPPLRYMGEDGCWYGMEIRPINEKEENMVKSLVSA

SEQ ID NO: 8 - protein, 352 aa, Dengue virus type 4
DMGCVASWSGKELKCGSGIFVVDNVHTWTEQYKFQPESPARLASAILNAHKDGVCGIRSTTRLENVMWKQITNELNYVLWEGGHDLTVVAGDVKGVLTKGKRALTPPVSDLKYSWKTWGKAKIFTPEARNSTFLIDGPDTSECPNERRAWNSLEVEDYGFGMFTTNIWMKFREGSSEVCDHRLMSAAIKDQKAVHADMGYWIESSKNQTWQIEKASLIEVKTCLWPKTHTLWSNGVLESQMLIPKSYAGPFSQHNYRQGYATQTVGPWHLGKLEIDFGECPGTTVTIQEDCDHRGPSLRTTTASGKLVTQWCCRSCTMPPLRFLGEDGCWYGMEIRPLSEKEENMVKSQVTA

SEQ ID NO: 9 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 10 - protein, 449 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 11 - protein, 285 aa, Neisseria gonorrhoeae
MTRSKPVNRTTFCCLSLTAGPDSDRLQQRRGGGGGVAADIGTGLADALTAPLDHKDKGLKSLTLEASIPQNGTLTLSAQGAEKTFKAGGKDNSLNTGKLKNDKISRFDFVQKIEVDGQTITLASGEFQIYKQDHSAVVALRIEKINNPDKIDSLINQRSFLVSDLGGEHTAFNQLPDGKAEYHGKAFSSDDADGKLTYTIDFAAKQGHGKIEHLKTPEQNVELASAELKADEKSHAVILGDTRYGGEEKGTYRLALFGDRAQEIAGSATVKIGEKVHEIGIADKQ